(19) United States
(12) Patent Application Publication
(10) Pub. No.: US 2011/0144044 A1
(43) Pub. Date: Jun. 16, 2011

(54) Composition and method for increasing post workout recovery

(75) Inventors: Michael Kichuk, Toronto (CA); Jason Bell, Toronto (CA); Stephen Brown, Toronto (CA); Shan Chaudhuri, Toronto (CA)

(73) Assignee: Allmax Nutrition Inc.

(21) Appl. No.: 12/654,193

(22) Filed: Dec. 14, 2009

(51) Int. Cl. A61K 31/704; A61K 31/715

(52) U.S. Cl. 514/26; 514/60

(57) ABSTRACT

The composition of a dietary supplement is provided for increasing post workout recovery, increasing replenishment and regaining energy following exercise. A method for achieving the same is also provided. The present composition comprises branched chain amino acids, caffeine and a source of carbohydrates including waxy maize. The dietary supplement may further comprise one or more of Ginseng providing ginsenoside saponins, and Fenugreek seeds providing 4-Hydroxyisoleucine.

### FIELD OF THE INVENTION

The present invention concerns compositions and methods for increasing post workout recovery, increasing replenishment and for regaining energy following exercise.

### BACKGROUND OF THE INVENTION

Fatigue during exercise is a common experience irrespective of the level of activity and it is manifested as a decrease in exercise tolerance. Fatigue during prolonged exercise is associated with the depletion of skeletal muscle glycogen concentration, as has been shown in experiments using a percutaneous needle biopsy technique to obtain samples of human muscle. (Clyde Williams, Department of Physical Education and Sports Science, University of Technology, Loughborough).

Researchers from the University of South Carolina have studied the effects of intra-cerebro ventricular injection of caffeine and the adenosine A1 and A2 receptor agonist, 5′-N-ethylcarboxamidoadenosine (NECA) on treadmill run time to fatigue in rats. NECA was chosen for the study because caffeine is a nonselective adenosine receptor antagonist, and it is not known which of the four subtypes of adenosine receptors may be involved in an effect of caffeine on fatigue. However, A2b and A3 receptors are relatively less active than A1 and A2a receptors under normal physiological conditions. Central Nervous system (CNS) administration of caffeine does increase treadmill run time to fatigue in rats by approximately 60 percent. CNS administration of the selective adenosine A1 and A2 receptor agonist NECA was found to significantly reduced run time to fatigue. (“Central Nervous System Effects of Caffeine and Adenosine on Fatigue,” are J. Mark Davis, Zuowei Zhao, Howard S. Stock, Kristen A. Mehl, James Buggy, and Gregory A. Hand, all from the Schools of Public Health and Medicine, University of South Carolina, Columbia, S.C.).

Researchers at the University of Guelph conducted a study that examined the possible effects of caffeine ingestion on muscle metabolism and endurance during intense exercise. They tested 14 subjects after they ingested either a placebo or caffeine (6 mg/kg) with an exercise protocol in which they cycled for 2 min, rested 6 min, cycled 2 min, rested 6 min, and then cycled to voluntary exhaustion. In each exercise the intensity required the subject's maximal O₂ consumption. Subjects had muscle and venous blood samples taken before and after each exercise period. The caffeine ingestion resulted in a significant increase in endurance. This study examined the impact of caffeine ingestion on muscle glycogen and lactate metabolism during intense exercise when power output was controlled, as well as the effect on endurance during intense exercise performed until voluntary exhaustion. The major findings showed that after caffeine ingestion, both muscle lactate and plasma epinephrine concentrations were increased even though the power output, the total work done, and net muscle glycogenolysis were unaffected. In addition, exercise endurance was enhanced by caffeine under circumstances when muscle glycogen availability was not a limiting factor. Caffeine ingestion resulted in a significant increase in endurance. (Jackman, M., P. Wendling, D. Friars, and T. E. Graham. Metabolic, catecholamine, and endurance responses to caffeine during intense exercise. J. Appl. Physiol. 81(4): 1658-1663, 1996).

Branched chain amino acid (BCAA) catabolism in skeletal muscle is regulated by the branched-chain a-keto acid dehydrogenase (BCKDH) complex, located at the second step in the BCAA catabolic pathway. Exercise activates the muscle BCKDH complex, resulting in enhanced BCAA catabolism. Therefore, exercise increases the BCAA requirement. BCAA supplementation before exercise attenuates the breakdown of muscle proteins during exercise in humans and that BCAA strongly promotes protein synthesis in skeletal muscle in humans and rats, suggesting that a BCAA supplement may attenuate muscle damage induced by exercise and promote recovery from the damage.

A clinical study examined the effects of BCAA supplementation on delayed-onset muscle soreness (DOMS) and muscle fatigue induced by squat exercise in humans. The results obtained showed that BCAA supplementation prior to squat exercise decreased DOMS and muscle fatigue occurring for a few days after exercise. These findings suggest that BCAAs is useful for muscle recovery following exercise. (Nutraceutical Effects of Branched-Chain Amino Acids on Skeletal Muscle—Yoshiharu Shimomura, Yuko Yamamoto, Gustavo Bajotto, Juichi Sato, Taro Murakami, Noriko Shimomura, Hisamine Kobayashi, and Kazunori Mawatari—Nutr. 136: 529S-532S, 2006).

Several factors have been identified to cause peripheral fatigue during exercise, whereas the mechanisms behind central fatigue are less well known. Change in the brain 5-hydroxytryptamine (5-HT) level is one factor that has been suggested to cause fatigue. The rate-limiting step in the synthesis of 5-HT is the transport of tryptophan across the blood-brain barrier. This transport is influenced by the fraction of tryptophan available for transport into the brain and the concentration of the other large neutral amino acids, including the BCAAs, which are transported via the same carrier system.

Studies in human subjects have shown that the plasma ratio of free tryptophan (unbound to albumin)/BCAAs increases and that tryptophan is taken up by the brain during endurance exercise, suggesting that this may increase the synthesis of 5-HT in the brain. Ingestion of BCAAs increases their concentration in plasma. This may reduce the uptake of tryptophan by the brain and also 5-HT synthesis and thereby delay fatigue.

Sustained exercise leads to increases in the plasma-concentration ratio of free tryptophan/BCAAs, an uptake of tryptophan by the brain in humans, and an increase in the synthesis and release of 5-HT in the rat brain. Elevated levels of brain 5-HT may contribute to the development of central fatigue during and after sustained exercise. Intake of BCAAs increases their concentration in plasma and prevents the increase in free tryptophan/BCAAs, which according to the hypothesis should decrease the synthesis of 5-HT in the brain and delay central fatigue. Support for this is presented in some studies, where intake of BCAAs was reported to decrease mental fatigue and improve mental agility as well as improve physical performance.

Accordingly, when BCAAs were supplied to human subjects during a standardized cycle ergometer exercise their ratings of perceived exertion and mental fatigue were reduced, and, during a competitive 30-km cross-country race, their performance on different cognitive tests was improved after the race. In some situations the intake of BCAAs also improves physical performance. The results also suggest that ingestion of carbohydrates during exercise delays a possible effect of BCAAs on fatigue since the brain's uptake of tryptophan is reduced. (A Role for Branched-Chain Amino Acids in Reducing Central Fatigue by Eva Blomstrand. J. Nutr. 136:544 S-547S, February 2006).

A study was conducted to determine the effect of carbohydrate ingestion and environmental heat on the development of fatigue and the distribution of power output during a cycling time trial. Cyclists performed four 90-min constant-pace cycling trials at 80% of second ventilatory threshold. The aims of this study were to examine the effects of environmental heat stress and carbohydrate consumption on the distribution of power output during a self-paced cycling time trial performed immediately following prolonged moderate-intensity cycling.

The important findings of this study were that carbohydrate ingestion attenuated the rise in serum free fatty acid concentration but did not influence percent muscle activation following exercise; carbohydrate ingestion improved time trial performance in hot but not temperate conditions; exercise in high ambient temperature resulted in an increase in serum prolactin concentration and a reduction in percent activation of the quadriceps; and performance in a high ambient temperature was associated with a relatively high power output at the beginning of the time trial, whereas carbohydrate ingestion resulted in a greater increase in power output during the final section of the time trial. (Chris R. Abbiss, Jeremiah J. Peiffer, Jonathan M. Peake, Kazunori Nosaka, Katsuhiko Suzuki, David T. Martin, and Paul B. Laursen. J Appl Physiol 104: 1021-1028, 2008.)

Another study was conducted on roles of extended release carbohydrate, for example, Waxy Maize, or Short-Chain Fatty Acids (SCFA). Waxy maize is starch that resists small intestinal digestion and enters the large bowel in normal humans. In vitro studies have shown that incubation with dilate colonic resistance arterioles in isolated human colonic segments. The mechanisms of action involve local neural networks as well as chemo receptors together with direct effects on smooth muscle cells. Rectal infusion of SCFA into human surgical patients leads to 1.5- to 5.0-fold greater blood flow. It is expected that greater blood flow enhances tissue oxygenation and transport of absorbed nutrients. Extended release carbohydrate thus is likely to further help recover from fatigue by increasing blood flow to muscle cells. (David L. Topping and Peter M. Clifton. Physiol. Rev. 81: 1031-1064, 2001)

In view of the above, it is of interest to further examine synergistic effects of caffeine with carbohydrate and amino acid, on human fatigue and post workout recovery.

### SUMMARY OF THE INVENTION

In a first embodiment, the present invention relates to a supplemental dietary composition comprising branched chain amino acids, a source of caffeine and extended release carbohydrates including waxy maize.

In a second embodiment, the present invention relates to a method for optimizing muscle glycogen levels and increasing muscle lactate and plasma ephedrine levels. The method comprises selecting a supplemental dietary composition comprising branched chain amino acids, a source of caffeine and extended release carbohydrates, and orally administering to a human an effective amount of said composition.

### DETAILED DESCRIPTION OF THE INVENTION

The present invention, according to various embodiments thereof, provides compositions and methods for aiding in post-glycogen restorations, promoting endurance and enhancing motor performance, increasing serum ammonia levels during exercise; decreasing muscle breakdown and inhibiting muscle glycogen degradation during exercise.

Branched chain amino acids, a source of caffeine and extended release carbohydrates have been found to aid in increasing post workout recovery, increasing replenishment and regaining energy following exercise.

In a preferred embodiment, the present invention employs the use of branched chain amino acids, a source of caffeine and a slow digestible, waxy starch. The main characteristic of waxy starches is that they generally contain a large amount of a highly branched chain starch called amylopectin that is slowly-digestible. Waxy maize starch is one example of a waxy starch, commonly used for the purposes of post-glycogen restorations.

Caffeine promotes alertness and wakefulness, enhances cognitive performance, relieves fatigue, promotes endurance and enhances motor performance.

Amino acids aid in muscle protein synthesis and it is stimulated in the recovery period after resistance exercise. The oral administration of branched-chain amino acids before exercise increases serum ammonia levels during exercise; decreases muscle breakdown and inhibits muscle glycogen degradation during exercise. Orally administered essential amino acids (EAAs) stimulate net muscle protein balance in healthy volunteers when consumed after resistance exercise.

Post exercise muscle glycogen synthesis is an important factor in determining the time needed to recover from prolonged exercise. The reliance on muscle glycogen increases with increasing exercise intensity and a direct relation between fatigue and depletion of muscle glycogen stores. Therefore, the post exercise glycogen synthesis rate is an important factor in determining the time needed to recover. To optimize glycogen synthesis rates, adequate amounts of carbohydrate and amino acids should be ingested. This aids in post-glycogen restorations and promoting endurance and enhances motor performance, increasing serum ammonia levels during exercise; decreasing muscle breakdown and inhibiting muscle glycogen degradation during exercise. Caffeine ingestion increase both muscle lactate and plasma epinephrine concentrations thereby increasing post workout recovery, increasing replenishment and regaining energy following exercise.

The independent and combined effects of a balanced mixture of amino acids (i.e., EAAs+NEAAs) and carbohydrate on muscle protein synthesis after resistance exercise has been studied. The response of muscle protein to the bolus ingestion of a drink containing essential amino acids and carbohydrate after resistance exercise has also been studied. A three-compartment model was used to determine the kinetics of leg muscle protein. It was concluded that essential amino acids with carbohydrates stimulate muscle protein anabolism by increasing muscle protein synthesis when ingested 1 or 3 h after resistance exercise. (Elisabet Borsheim, Kevin D. Tipton, Steven E. Wolf, and Robert R. Wolfe Am J Physiol Endocrinol Metab 283: E648-E657, 2002).

Although the present invention is not to be limited by any theoretical explanation, it is hypothesized that depletion in muscle glycogen stores is the primary factor that sets in fatigue after workout and further that carbohydrate and amino acid in adequate amounts optimize muscle glycogen levels. Caffeine ingestion is known to increases muscle lactate and plasma ephedrine levels. Therefore, the present inventors consider restoration of muscle glycogen levels and increase in muscle lactate and plasma ephedrine levels lead to an increase in post workout recovery, increase replenishment and regain energy following exercise.

The composition may further comprise one or more of the following: Ginseng providing ginsenoside saponins, and Fenugreek seeds providing 4-Hydroxyisoleucine. Most preferably the composition is in the nature of a dietary supplement.

In a further embodiment, the present invention further relates to a method of optimizing muscle glycogen levels and increasing muscle lactate and plasma ephedrine levels by administering to a human an effective amount of the supplemental dietary compositions according to the present invention. Such administration acts to increase post workout recovery, increase replenishment and regain energy following exercise. The compositions and methods of the present invention are particularly advantageous for athletes and bodybuilders to enhance performance. The effective amount of the present composition administered to the athlete varies depending on the desired effect, the body weight and characteristics of the athlete, and the like. For example, in preferred embodiments, the subject supplemental dietary compositions are administered to the diet of the athlete or bodybuilder on a daily basis.

In a further preferred embodiment, the present compositions can be administered in the form of a powder that is dissolvable in water and consumed immediately thereafter or other suitable beverage, a gel or gelatin, or gel or gelatin powdered mixture, pudding or pudding powdered mixture.

Although the following examples illustrate the practice of the present invention in some of its embodiments, the examples should not be construed as limiting the scope of the invention. Other embodiments will be apparent to one skilled in the art from consideration of the specification and examples. Table 1 illustrates on sample composition for the supplemental dietary composition of the present invention.

TABLE 1

| | Amount | |
|---|---|---|
| | per dose | % Dietary |
| Ingredients | (mg) | Ingredients |
| Sucrose | 9375 | 16.51% |
| Glucose | 9375 | 16.51% |
| Fructose | 9375 | 16.51% |
| Waxy Maize | 9375 | 16.51% |
| Whey Protein Concentrate | 9000 | 15.85% |
| Hydrolyzed Whey Protein | 2192 | 3.86% |
| Caesin | 2192 | 3.86% |
| L-Isoleucine | 1227 | 2.16% |
| L-Valine | 1023 | 1.80% |
| Alpha ketoisocaproate | 125 | 0.22% |
| Caffeine | 50 | 0.09% |
| Glutamine | 150 | 0.26% |
| Ginseng | 60 | 0.11% |
| Octacosanol | 5 | 0.01% |
| Inositol | 150 | 0.26% |
| Fenugreek seeds (supplying | 50 | 0.09% |
| 4-Hydroxyisoleucine) | | |
| Flavors and Maskers | 2000 | 3.52% |
| Disodium Phosphate | 200 | 0.35% |
| Dipotassium Phosphate | 200 | 0.35% |
| Calcium Chloride | 125 | 0.22% |
| Magnesium Cyclinate | 100 | 0.18% |
| Citric and/or Malic Acid | 175 | 0.31% |
| Vitamin C as Ascorbic Acid | 100 | 0.18% |
| Coenzyme Q10 | 50 | 0.09% |
| Vitamin E Mixed Tochopherols | 100 | 0.18% |
| Folic Acid | 0.6 | 0.0011% |
| Vitamin B12 | 0.0857 | 0.0002% |
| | 56774.69 | 100.00% |

The invention has been described with specific embodiments and examples thereof; however, it will be evident that various modifications and changes may be made thereto without departing from the broader spirit and scope of the invention.

What is claimed:

1. A supplemental dietary composition for optimizing muscle glycogen levels and increasing muscle lactate and plasma ephedrine levels, the composition comprising branched chain amino acids, a source of caffeine and extended release carbohydrates.

2. The supplemental dietary composition of claim 1 wherein the carbohydrates are waxy starches.

3. The supplemental dietary composition of claim 2, wherein the waxy starches are waxy maize.

4. The supplemental composition of claim 1, further comprising Fenugreek seeds supplying 4-Hydroxyisoleucine.

5. The supplemental composition of claim 1, further comprising Ginseng supplying Ginsenoside saponins.

6. The supplemental composition of claim 4, further comprising Ginseng supplying Ginsenoside saponins.

7. A method for optimizing muscle glycogen levels and increasing muscle lactate and plasma ephedrine levels, said method comprising the steps of: selecting a supplemental dietary composition comprising branched chain amino acids, a source of caffeine and extended release carbohydrates; and orally administering to a human an effective amount of said composition.

8. The method of claim 7, wherein the extended release carbohydrates are waxy starches.

9. The method of claim 8, wherein the waxy starches are waxy maize.

10. The method of claim 1, wherein the supplemental dietary composition further comprises Fenugreek seeds supplying 4-Hydroxyisoleucine.

11. The method of claim 1, wherein the supplemental dietary composition further comprises Ginseng supplying Ginsenoside saponins.

12. The method of claim 10 wherein the supplemental dietary composition further comprises Ginseng supplying Ginsenoside saponins.
